(19) United States
(12) Patent Application Publication
(10) Pub. No.: US 2014/0045904 A1
(43) Pub. Date: Feb. 13, 2014

(54) NIACIN FORMULATIONS AND METHODS WITH REDUCED FLUSHING SIDE EFFECT

(71) Applicant: Eric Hauser Kuhrts, Bodega, CA (US)

(72) Inventor: Eric Hauser Kuhrts, Bodega, CA (US)

(21) Appl. No.: 13/878,670

(22) PCT Filed: Oct. 10, 2011
(86) PCT No.: PCT/US11/55595

§ 371 Date: Sep. 23, 2013

Related U.S. Application Data
(60) Provisional application No. 61/391,669, filed on Oct. 10, 2010.

(51) Int. Cl. A61K 31/455; A61K 31/12

(52) U.S. Cl. CPC A61K 31/455; A61K 31/12
USPC 514/356; 514/685

(57) ABSTRACT

Compositions and methods for reducing the flushing effect (cutaneous erythema) of niacin and for treating hyper lipemia and elevated triglycerides, comprising a prenylflavonoid, such as xanthohumol, and niacin (nicotinic acid or Vitamin B3).

### BACKGROUND

Niacin is a drug that can be used for reducing plasma cholesterol and triglyceride levels and raising HDL-cholesterol levels and is also present in certain over the counter (OTC) and prescription drugs. Even with the availability of a wide-range of supplement and therapeutic approaches to lowering cholesterol, niacin is still considered to be one of the more clinically effective. Its effectiveness is due in large part to its ability to improve blood levels of key risk-associated plasma lipoproteins, e.g., it lowers LDL-cholesterol, Lp₍ₐ₎ and triglycerides, while raising HDL-cholesterol. In this regard, it is notable that over 50% of coronary heart disease patients have low HDL (36%) or high Lp₍ₐ₎ (16%) levels and that the ratio of total plasma cholesterol to HDL-cholesterol and the Lp₍ₐ₎ levels are highly reliable predictors of cardiovascular risk. The ability to lower Lp₍ₐ₎-cholesterol and triglycerides and raise HDL-cholesterol is not found in most other cholesterol lowering products and is particularly significant in addressing diabetic dyslipidemia.

While fairly large doses of niacin (1-2 grams/day) are sometimes used to lower total and LDL cholesterol as well as triglycerides, low doses of niacin (300-1,000 mg/day) can raise HDL cholesterol up to 45%. Niacin also increases prostacyclin (PGI-2), a beneficial prostanglandin that is cardioprotective. Prostacyclin is synthesized in vascular endothelial cells and has vasodilative and fibrinolytic properties, as well as properties for inhibiting platelet aggregation, and triggering the outflow of free cholesterol from endothelial cells by enhancing the activity of acid cholesterol ester hydrolase, with the free cholesterol being transported away by HDL cholesterol. This in turn neutralizes the damaging effects of oxygen free radicals, and can prevent post-ischemic reperfusion injury, altering progressive damage that is the result of hypoxic injury to the cardiac myocytes.

The prostaglandins (PGs) are a family of structurally related molecules produced by cells in response to extrinsic stimuli. The prostaglandins also regulate cellular growth, homeostasis, and differentiation. PGs are derived mainly from the fatty acid arachidonate, and are released from membrane phospholipids by the action of phospholipases. Arachidonic acid is first converted to an unstable intermediate by cyclooxygenases and then to a series of prostaglandins, including prostaglandin D-2 (PGD-2), prostaglandin E-2 (PGE-2), prostaglandin F-2 (PGF-2), prostacyclin (PGI-2), and thromboxane A-2 (Txa-2), via the action of specific PG synthetases. PGD-2 is present in a variety of tissues and cells and has marked effects on platelet aggregation and nerve functions, as well as relaxation of vascular and nonvascular smooth muscles. Normally, PGD-2 is present in biological fluids in very small amounts (picomolar-to-nanomolar concentrations). When niacin is given orally (500 mg), levels of the major metabolite of PGD-2 increase from 400 to 800-fold within 12-45 minutes after ingestion, and then decline to near normal levels within 2-4 hours (Morrow, J. et al, Prostaglandins; 38, 2: 263-274; 1989).

Niacin is known to produce flushing or a redness of the skin, which is a type of inflammation. The source of this inflammation has been identified as a prostaglandin, namely prostaglandin D-2 (PGD-2). Interestingly, ingestion of oral niacin does not increase the major urinary metabolite of PGE-2 (PGE-M), but produced a 2-fold increase in prostacyclin (PGI-2). In addition, niacin causes an increase in PGI-2 synthesis in human whole blood in vitro. The flushing side effect produced by niacin is a major reason for the lack of compliance associated with its use as a drug for modulating cholesterol. Because high doses of niacin are often desired for use to effect cholesterol, patients are often told to titrate doses, starting at low doses and slowly increasing week by week, over about a 6 week period, until the higher doses can be tolerated. If a few days are missed, the patient must usually start the titration again and work their way back up to the higher doses. If niacin therapy is stopped, the tolerance to flushing is also often lost.

The metabolic pathway of PGD-2, the major prostanglandin increased by niacin, is believed to have been determined. PGD-2 converts into cyclopentenone prostaglandins of the J series, primarily PGJ2, delta12-PGJ2, and 15-deoxy-delta 12,14-PGJ2 (15d-PGJ2). The later metabolite, 15d-PGJ2, is a potent endogenous PPARγ agonist (PPARγ ligand). PPARγ is a transcription factor for a number of genes involved in lipid metabolism, and recently, has been the focus of a class of antidiabetic drugs called TZDs (thiazolidinediones). Some of these drugs have demonstrated significant cardiovascular benefit, in addition to, and independent of, their ability to control glucose.

While niacin has been shown to increase endogenous production of PGD-2, and its metabolites, it does not appear to significantly increase PGE-2. Niacin, therefore, is a selective prostaglandin stimulator. Recent research has demonstrated that cyclooxygenase-1 (COX-1) is responsible for producing PGD-2, while cyclooxygenase-2 (COX-2) is primarily responsible for producing PGE-2. This was further verified by the fact that aspirin, which inhibits both COX-1 and COX-2, can suppress the flush from oral niacin, by suppressing production of PGD-2, whereas a selective COX-2 inhibitor such as rofecoxib, does not completely suppress flushing. This indicates that the production of PGD-2 and the corresponding flushing of the skin is primarily mediated by production of PGD-2 by COX-1 instead of COX-2. Additional evidence of this comes from in vitro cell line data in rat macrophages, in which induction of COX-2 resulted in the preferred production of PGE-2, and PGD-2 production was controlled by COX-1 (Matsumoto, H. et al; Biochem Biophys Res Commun; 1997; 230 (1): 110-114). When a dual inhibitor (indomethacin) of COX-1 and COX-2 was used, endogenous production of both prostanoids, PGE-2 and PGD-2 were suppressed, whereas when the cells where incubated with a selective COX-2 inhibitor, PGE-2 was the primary prostanoid that was reduced. Likewise, when the cells where stimulated by LPS to induce COX-2, indomethacin produced a reduction in both PGD-2 and PGE-2, but the selective COX-2 inhibitor primarily reduced PGE-2 production, but spared PGD-2 production. Therefore, there is evidence to suggest that stimulation of in vivo PGD-2 prostanoid production by oral niacin is primarily a COX-1 phenomenon, and that selective COX-2 inhibitors will not result in significant reduction of PGD-2.

Further evidence to support the selective production of PGD-2 by COX-1 was evidenced by giving subjects an oral dose of niacin after pretreatment with the selective COX-2 inhibitor rofecoxib. Rofecoxib inhibits COX-2 by about 90% in vivo, while sparing COX-1. Pretreatment with rofecoxib did not result in a reduction of flushing from a single dose of 500 mg of immediate-release niacin. Since the flushing can be reduced by cumulative pretreatment with aspirin, which also inhibits COX-1, this provides further evidence that PGD-2 production by niacin is primarily by COX-1 as opposed to COX-2.

Niacin induced flushing also appears to be a biphasic phenomena, with the first peak in intensity occurring shortly after the start of the reaction, and the second peak after the first has subsided.

Although niacin has been used for more than 50 years to lower cholesterol levels, we are only now starting to piece together the molecular mechanism by which it does this, following the discovery that G protein-coupled receptor 109A (GPR109A) is a receptor for niacin that mediates some of its myriad effects. GPR109A is highly expressed in adipocytes, where stimulation results in the profoundly antilipolytic effect of niacin and, in turn, the acute reduction in levels of plasma free fatty acids.

Since the most common side-effect associated with niacin therapy is skin toxicity, it would be desirable to have a formulation of niacin without the side effects and tedious dosing issues that limit its widespread use. Despite its ability to prevent heart attacks (MI), in practice, niacin lacks widespread use because of its aggressive irritant effects in the skin. Approximately 20% or more of niacin-treated subjects drop out of clinical trials, and in practice, nonadherence and discontinuation are important obstacles to niacin therapy. In niacin exposure, these features are transient and are more akin to an acute hypersensitivity syndrome.

Given the high rates of discontinuation, development of a clinical strategy to suppress niacin-associated skin toxicity without compromising the benefits of the drug if it is to be used to prevent heart attacks on a broader scale would be an advancement in the art. As with the effects on lipid levels, the mechanism underlying niacin-associated skin toxicity has been elusive. More recently, the identification of GPR109A as a receptor for niacin has revolutionized the study of the noxious skin effects of the drug. GPR109A is highly expressed in a variety of cells, notably neutrophils, adipocytes, Langerhans cells, keratinocytes, and monocytes, and growing evidence suggests that agonizing GPR109A on skin immune cells incites a cascade of events that drives the flushing, if not the other irritative features. Upon stimulation of immune cells, GPR109A promotes activation of phospholipase A2, production of arachidonic acid, and, via cyclooxygenase (COX) enzymes, generation of prostaglandin D2 (PGD2) and possibly other prostanoids. These activate DP1 and EP2/4 receptors, respectively, cause relaxation of vascular smooth muscle and, in turn, vasodilation and flushing. Other candidates of niacin-associated skin toxicity include Langerhans cells, macrophages, mast cells, and platelets.

A working theory points to the Langerhans cell/PGD2/DP1 pathway as the prime suspect for flushing, and this pathway has been targeted for discovery and drug therapy. A DP1 receptor antagonist, laropiprant, has been developed to mitigate niacin-induced flushing, and is currently marketed in Europe. In clinical trials, laropiprant substantially reduced, but fell far short of eliminating, objectively measured flushing and other subject-reported skin symptoms. This suggests that, in humans, other pathways beyond the PGD2/DP1 pathway may also contribute to flushing. These results are consistent with a mouse model of niacin-induced flushing in which mice lacking the DP1 receptor had only a partial reduction in flushing. These studies led to the hypothesis that niacin-associated skin toxicity, including flushing, is multi-factorial and that factors beyond PGD2 may be involved in vasodilation and the other skin effects of the drug. Although much progress has been made, the complete details of how niacin causes flushing and niacin-associated skin toxicity still remain somewhat of a mystery.

### SUMMARY

This disclosure relates to unique pharmaceutical compositions of niacin with reduced flushing side-effects comprising niacin or nicotinic acid and a prenylflavonoid, such as for example, a prenylflavonoid derived from hops (humulus Lupulis L.), xanthohumol. Surprisingly, it has been discovered that xanthohumol and other similar prenylflavonoids significantly reduce the flushing side-effects associated with niacin therapy for cholesterol or elevated tryglycerides.

In one embodiment, a pharmaceutical composition for administration of hypolipemic and/or hypotryglyceridemic amounts of niacin having reduced capacity to provoke a flushing reaction in a subject. The composition can comprise a hypolipemic or hypotryglyceridimemic amount of niacin, and a prenylflavonoid. The prenylflavonoid can be selected from the group consisting of xanthohumol, xanthogalenol, desmethylxanthohumol (2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O -methylxanthohumol, 6-geranylnaringenin, 8-geranylnaringenin, metabolites thereof, derivatives thereof, and combinations thereof.

In another embodiment, a composition for treating dyslipidemia and/or hypertryclyceridemia can comprise an effective amount of niacin to treat dyalipidemia or hypertryglyceridemia, and xanthohumol. The xanthohumol can be present in an amount effective to reduce cutaneous erythma caused by the effective amount of niacin.

In another embodiment, a pharmaceutical composition for administration of HDL elevating or hypotriglyceridemic amounts of niacin having reduced capacity to provoke a flushing reaction in a subject can comprise water soluble xanthohumol and sustained-release niacin.

In another embodiment, a method of reducing or shortening the early titration phase of niacin dosing can comprise multiple steps. For example a first dose can be accelerated to at least 500 mg of sustained-release or immediate release niacin on a first day, and subsequent titrations can be advanced to 1,000 mg of niacin on a second day and 1,500 mg on a third day. On the first day, a pre-dose of xanthohumol can be administered at least 30 minutes prior to the first dose of niacin, and thereafter, prior to or concurrently with higher doses of niacin.

In some detail with respect to certain specific embodiments described herein, the flush reducing pharmaceutical composition of niacin can comprise from of 10-3,000 mg of niacin and from 1-500 mg of xanthohumol. This being stated, a unique feature of the present disclosure can be in the ability of the described formulations to eliminate the titration phase of niacin therapy, and/or enable a subject to immediately start niacin therapy with at least a 500-1,000 mg or more of immediate-release or sustained-release niacin on the first day of therapy. In addition, the flush reducing effect of xanthohumol is effective during both phases of the biphasic flushing reaction, especially during the second phase, which sometimes can occur many hours after ingestion of niacin.

### DETAILED DESCRIPTION

Before the present invention is disclosed and described, it is to be understood that this disclosure is not limited to the particular process steps and materials disclosed herein because such process steps and materials may vary somewhat. It is also to be understood that the terminology used herein is used for the purpose of describing particular examples only. The terms are not intended to be limiting because the scope of the present disclosure is intended to be limited only by the appended claims and equivalents thereof.

It is noted that, as used in this specification and the appended claims, the singular forms “a,” “an,” and “the” include plural referents unless the context clearly dictates otherwise.

As used herein “patient” or “subject” refers to a mammalian subject, including human.

As used herein “niacin” or “nicotinic acid” (also known as vitamin B-3) is pyridine-3-carboxylic acid, and can be identified under CAS number 59-67-6. The molecular formula is C₆H₅NO₂, and molar mass 123.11 g/mol. For purposes of clarification, niacin is not to be confused with niacinamide, the corresponding amide, which does not modulate cholesterol.

As used herein, “xanthohumol” (XN; 2′,4′,6′,4-tetrahydroxy-3′-prenylchalcone) includes the yellow-orange substance with a melting point of 172° C. and a molecular weight of 354.4. Xanthohumol is a prenylated chalcone or prenylflavonoid isolated from the hop plant (Humulus lupulus L.).

A “prenylflavonoid,” as used herein, refers to a prenylated compound having a substituted or unsubstituted phenol attached to a phenyl via a C₃ alkylene substituted with an oxo group. The C₃ alkylene may be present in a linear chain arrangement (e.g. a chalcone) or joined with other atoms to form a substituted or unsubstituted ring (e.g. a flavanone). Prenylflavonoids may be derived from natural sources (e.g. hops), or synthesized chemically. Tabat et al., Phytochemistry 46: 683-687 (1997).

As used herein, a “prenylated” compound refers to those compounds with an attached —CH₂—CH═C(CH₃)₂ group (e.g. geranylated compounds), optionally hydroxylated prenyl tautomers (e.g. —CH₂—CH—C(CH₃)═CH₂, or —CH₂—C(OH)—C(CH₃)═CH₂), and optionally hydroxylated circularized prenyl derivatives having the formula:

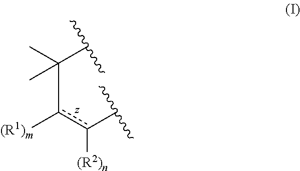

In Formula (I), the dashed bond z represents a double bond or a single bond. R¹ and R² are independently hydrogen or OH. The symbol [symbol] represents the point of attachment to the remainder of the prenylated compounds.

Concentrations, amounts, solubility's, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a concentration range of 0.5 to 400 should be interpreted to include not only the explicitly recited concentration limits of 0.5 and 400, but also to include individual concentrations within that range, such as 0.5, 0.7, 1.0, 5.2, 8.4, 11.6, 14.2, 100, 200, 300, and sub-ranges such as 0.5-2.5, 4.8-7.2, 6-14.9, 55, 85, 100-200, 117, 175, 200-300, 225, 250, and 300-400, etc. This interpretation should apply regardless of the breadth of the range or the characteristic being described.

In accordance with the present disclosure, unique pharmaceutical compositions of niacin with reduced flushing side-effects comprising niacin (or nicotinic acid, vitamin B3) and a prenylflavonoid, such as for example, a prenylflavonoid derived from hops (humulus Lupulis L.), such as xanthohumol. Surprisingly, it has been discovered that xanthohumol and other similar prenylflavonoids significantly reduce the flushing side-effects associated with niacin therapy for cholesterol or elevated tryglycerides.

In one embodiment, a pharmaceutical composition for administration of hypolipemic and/or hypotryglyceridemic amounts of niacin having reduced capacity to provoke a flushing reaction in a subject. The composition can comprise a hypolipemic or hypotryglyceridimemic amount of niacin, and a prenylflavonoid. The prenylflavonoid can be selected from the group consisting of xanthohumol, xanthogalenol, desmethylxanthohumol (2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O -methylxanthohumol, 6-geranylnaringenin, 8-geranylnaringenin, metabolites thereof, derivatives thereof, and combinations thereof.

In another embodiment, a composition for treating dyslipidemia and/or hypertryclyceridemia can comprise an effective amount of niacin to treat dyalipidemia or hypertryglyceridemia, and xanthohumol. The xanthohumol can be present in an amount effective to reduce cutaneous erythma caused by the effective amount of niacin.

In another embodiment, a pharmaceutical composition for administration of HDL elevating or hypotriglyceridemic amounts of niacin having reduced capacity to provoke a flushing reaction in a subject can comprise water soluble xanthohumol and sustained-release niacin.

In another embodiment, a method of reducing or shortening the early titration phase of niacin dosing can comprise multiple steps. For example a first dose can be accelerated to at least 500 mg of sustained-release or immediate release niacin on a first day, and subsequent titrations can be advanced to 1,000 mg of niacin on a second day and 1,500 mg on a third day. On the first day, a pre-dose of xanthohumol can be administered at least 30 minutes prior to the first dose of niacin, and thereafter, prior to or concurrently with higher doses of niacin.

In one aspect, the present disclosure provides a highly soluble formulation including a prenylflavonoid such as xanthohumol, a non-ionic surfactant or a cyclodextrin, and niacin.

Hops (Humulus lupulis L.) has been used for centuries as a bittering agent in the brewing of beer. Hops contains alpha acids such as humulone, co-humuone, ad-humulone, and beta acids such as lupulone and co-lupulone. Hops also contains many flavonoids, such as xanthohumol, isoxanthohumol, desmethylxanthohumol, 8-prenylnaringenin, and 6-prenylnaringenin. Xanthohumol is a yellow-orange substance with a melting point of 172° C. and a molecular weight of 354.4. A typical ethanol extract of hops yields about 3 mg/g (3 wt %) of xanthohumol out of a total flavonoid content of 3.46 mg/g. Dried hop contains about 0.2 to 1.0 wt % xanthohumol, but can be extracted and purified to very high concentrations, e.g., 98-99%.

Ethanol can be used to extract higher levels of the prenylflavonoids from hops. One exemplary typical prenylflavonoid content of an ethanol extract of hops includes xanthohumol (3 mg/g), desmethylxanthohumol (0.34 mg/g), isoxanthohumol (0.052 mg/g), 6-prenylnaringenin (0.061 mg/g), and 8-prenylnaringenin 0.015 (mg/g). Supercritical carbon dioxide extractions tend to contain much lower levels, or non-existent levels of, prenylflavonoids. In fact, these compounds are almost non-existent in standard CO₂ extracts because the prenylflavonoids are virtually insolvent on carbon dioxide. In the examples provided herein, a xanthohumol extract of purity of 98% has been used.

Prenylflavonoids useful in the present disclosure include prenylchalcones and/or prenylflavanones. In some embodiments, the prenylflavonoid is selected from the group consisting of xanthohumol, xanthogalenol, desmethylxanthohumol (2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O -methylxanthohumol, 6-geranylnaringenin, 8-geranylnaringenin, metabolites thereof, derivatives thereof, and combinations thereof. In certain embodiments, the prenylflavonoid is xanthohumol, a xanthohumol metabolite, or a xanthohumol derivative. In another embodiment, the prenylflavonoid is xanthohumol.

The prenylflavonoid may be derived from a natural source, such as hops. Prenylflavonoids may be isolated from hops through purification, fractionation, or separation methods that are known to those skilled in the art.

The amount of xanthohumol sufficient to reduce flushing levels may be from about 0.5 mg to about 1000 mg, from about 1 mg to about 500 mg, from about 5 mg to 250 mg, or from about 10 mg to 50 mg. In another example, the amount can be from about 1 mg to about 20 mg, or about 3 mg to about 10 mg. In some embodiments, the dose of xanthohumol can be 1 mg, 3 mg, 5 mg, 10 mg, or 20 mg, or 50 mg. In still other embodiments, the dose of xanthohumol can be about 5 mg. The xanthohumol is typically administered as a twice per day formulation, or more alternatively, as a once per day formulation. It is noted that though these values are given specifically for xanthohumol, they can be equally applicable to other prenylflavonoids and apply to those other compounds as well.

In some embodiments, the xanthohumol can be present in the niacin formulation at from 1 wt % to 50 wt %, or even more, or alternatively from 0.01 wt % to 25 wt %. For example, the xanthohumol can be present at concentrations of at least 1 wt %, 5 wt %, 10 wt %, 20 wt %, 25 wt %, 30 wt %, 35 wt %, 45 wt %, 45 wt %, or 50 wt %. In other embodiments the xanthohumol can be present in the niacin formulation a concentration from 0.01 wt % to 80 wt %, 5 wt % to 50 wt %, 10 wt % to 35 wt %, or 20 wt % to 25 wt %. The xanthohumol may also be present (e.g. in a tablet formulation) at a concentration from 0.5 to 50 mg per tablet. In other embodiments, the prenylflavonoid is present at a concentration from 0.01 mg/ml to 25 mg/tablet.

The niacin component or dose may be from at least 50 mg, 100 mg, 250 mg, 500 mg, 1,000 mg, 2,000 mg, or 3,000 mg in a single dose (a single dose can be one or multiple tables or capsules, or other dosage form). The niacin can be in sustained-release or immediate-release form. In some embodiments, the niacin and xanthohumol are in the same dosage form. In other embodiments, the niacin and xanthohumol are in separate dosage forms (two tablets, one of niacin and one of xanthohumol would still be considered separate dosage forms). In still further embodiments, the xanthohumol is consumed before the niacin, and in some cases, the xanthohumol is consumed up to 30 minutes, 1 hour, or 2 hours before the niacin.

In other embodiments, at least 0.5 mg, 1 mg, 2 mg, 3 mg, 4 mg, 5 mg, 10 mg, 20 mg, 30 mg, 40 mg, 50 mg, 100 mg, 200 mg, 300 mg, 400 mg, 500 mg, or 1 g of prenylflavonoid is present in the water soluble formulation. In other embodiments, 0.1 mg to 2 g, 0.5 mg to 1 g, 1 mg to 500 mg, 1 mg to 100 mg, 1 mg to 50 mg, 1 mg to 10 mg, or 1 mg to 5 mg of prenylflavonoid is present in the water soluble formulation.

In some embodiments, a water soluble prenylflavonoid formulation is in the form of a pharmaceutical composition. The pharmaceutical composition may include a prenylflavonoid, such as xanthohumol, other prenylflavonoid or prenylflavonoid metabolite or derivative thereof, a non-ionic surfactant, and a pharmaceutically acceptable excipient. Optionally, the formulation may include water. After a pharmaceutical composition including a prenylflavonoid has been formulated in an acceptable carrier, it can be placed in an appropriate container and labeled for treatment of an indicated condition. For administration of prenylflavonoids, such labeling would include, e.g., instructions concerning the amount, frequency, and method of administration. In one embodiment, the disclosure provides for a kit for the treatment of cholesterol problems in a human which includes a prenylflavonoid and instructional material teaching the indications, dosages, and schedule of administration of the prenylflavonoid.

Any appropriate dosage form is useful for administration of the formulations of the present disclosure, such as oral, parenteral and topical dosage forms, can be used. Oral preparations include tablets, pills, powder, dragees, capsules (e.g. soft-gel capsules), liquids, lozenges, gels, syrups, slurries, beverages, suspensions, etc., suitable for ingestion by the patient. The formulations of the present disclosure can also be administered by injection, that is, intravenously, intramuscularly, intracutaneously, subcutaneously, intraduodenally, or intraperitoneally. Additionally, the formulations of the present invention can be administered transdermally. The formulations can also be administered by in intraocular, intravaginal, and intrarectal routes including suppositories.

For preparing pharmaceutical compositions from the formulations of the present disclosure, pharmaceutically acceptable carriers can be either solid or liquid. Solid form preparations include powders, tablets, bilayer tablets or capsules, pills, capsules, cachets, suppositories, and dispersible granules. A solid carrier can be one or more substances, which may also act as diluents, flavoring agents, binders, preservatives, tablet disintegrating agents, or encapsulating materials. Details on techniques for formulation and administration are well described in the scientific and patent literature, see, e.g., the latest edition of Remington's Pharmaceutical Sciences, Maack Publishing Co, Easton Pa. (“Remington's”).

Suitable carriers include magnesium carbonate, magnesium stearate, talc, sugar, lactose, pectin, dextrin, starch (from corn, wheat, rice, potato, or other plants), gelatin, tragacanth, a low melting wax, cocoa butter, sucrose, mannitol, sorbitol, cellulose (such as methyl cellulose, hydroxypropylmethyl-cellulose, or sodium carboxymethylcellulose), and gums (including arabic and tragacanth), as well as proteins such as gelatin and collagen. If desired, disintegrating or co-solubilizing agents may be added, such as the cross-linked polyvinyl pyrrolidone, agar, alginic acid, or a salt thereof, such as sodium alginate. In powders, the carrier is a finely divided solid, which is in a mixture with the finely divided active component. In tablets, the active component is mixed with the carrier having the necessary binding properties in suitable proportions and compacted in the shape and size desired.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings (or other formulations) for product identification or to characterize the quantity of active compound (e.g., dosage), or for simply providing desired coloration. Pharmaceutical preparations of the present disclosure can also be used orally using, for example, push-fit capsules made of gelatin, as well as soft, sealed capsules made of gelatin and a coating such as glycerol or sorbitol. Push-fit capsules can contain prenylflavonoid mixed with a filler or binders such as lactose or starches, lubricants such as talc or magnesium stearate, and, optionally, stabilizers. In soft capsules, the prenylflavonoid compounds may be dissolved or suspended in suitable liquids, such as fatty oils, liquid paraffin, or liquid polyethylene glycol with or without stabilizers.

The formulations or compositions may be administered as a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. Regarding liquid preparations, solutions or suspensions can be prepared that are ready to use, or solids can be prepared that can be admixed with water in preparation for use.

The quantity of active component in a unit dose preparation may be varied or adjusted according to the particular application and the potency of the active component. The composition can, if desired, also contain other compatible therapeutic agents.

Thus, in accordance with embodiments of the present disclosure, the prenylflavonoids can be co-administered or preadministered (separately or as part of a common dosage form) with niacin at an amount suitable to at least partially alleviate flushing induced by the niacin in a majority of human subjects. In another embodiment, the prenylflavonoid can be present in an amount suitable to completely alleviate flushing induced by the niacin in a majority of human subjects.

### EXAMPLES

The following examples illustrate a number of variations of the present compositions, formulations, and methods that are presently known. However, it is to be understood that the following are only exemplary or illustrative of the application of the principles of the present disclosure. Numerous modifications and alternatives may be devised by those skilled in the art without departing from the spirit and scope of the present disclosure. The appended claims are intended to cover such modifications and arrangements. Thus, while the present compositions and methods have been described above with particularity, the following examples provide further detail in connection with what are presently deemed to be acceptable.

### Example 1

### Sustained-Release Niacin

Six human subjects were given a pre-dose of 10 mg. of xanthohumol in water made with 98% xanthohumol, and formulated to be water-soluble. This formulation had the following composition:

TABLE 1
Sustained-release niacin formulation with xanthohumol

| Ingredient | Wt % |
|---|---|
| Water | 95.68 |
| Glycerol Polyethylene Glycol Oxystearate | 5.01 |
| Xanthohumol (98%) | 0.22 |
| Citric Acid | 0.09 |

In this example, the xanthohumol is added to the warm (90° F.) glycerol polyethylene glycol oxystearate and mixed until dissolved and clear. The composition is then slowly added while mixing with warm water (90° F.) until dissolved (mixed for about 20 minutes). Citric acid is then added to adjust the pH to about 3.

This water soluble solution is added to water 60 minutes prior to dosing the subject with 1,000 mg of sustained-release niacin (Slo-Niacin, Upsher-Smith Laboratories, Minn. MN.). The amount of xanthohumol was thus 10 mg, and the dose of niacin was 1,000 mg. (e.g., two 500 mg tablets). Subjects were given the niacin 3 hours after the noon meal, prior to dinner. The subjects were asked to fill out questionnaires that evening and the next morning if they experienced any flushing, and rate the severity according to the chart below.

After 1 week of no xanthohumol or niacin treatment (wash-out), the same subjects were given 1,000 mg of the same sustained-release niacin, 3 hours after the noon meal, with plain water (placebo). Within 2-3 hours, 4 out of 6 subjects experienced moderate to severe flushing, and the other 2 subjects experienced mild flushing.

The flushing response due to niacin is a well known and characterized clinical event. The objective of this trial was to evaluate the effect of pre-treatment with xanthohumol as a means to reduce the flushing response. Results indicated that both intensity and duration of flushing were markedly reduced when subjects were pretreated with xanthohumol, as shown below in Table 3.

TABLE 2
Assessment of flushing

| | |
|---|---|
| Very Severe | Bright red face, neck and upper trunk, nausea/vomiting, |
| | fainting and needs medical attention. |
| Severe | Bright red facial area and upper trunk. Itchy and prickly |
| | feeling of skin. |
| Moderate | Light to patchy redness. Itching and tingling of face |
| | and/or upper trunk. |
| Mild | Slight itching and tingling feeling. |
| None | No reaction |

The incidence of the maximum flushing response post initial dosing as documented by the subjects is summarized below.

TABLE 3
Maximum Flushing Response Incidence Post Initial Dosing - Subjects

| | | | | | Very |
|---|---|---|---|---|---|
| | None | Mild | Moderate | Severe | Severe |
| Xanthohumol + SR | 3 | 2 | 0 | 0 | 0 |
| Niacin | | | | | |
| Placebo + | 0 | 0 | 2 | 3 | 0 |
| SR Niacin | | | | | |

### Example 2

### Immediate-Release Niacin

Three subjects were given 500 mg of immediate-release niacin (Goldline Laboratories, Sellersville, Pa., niacin, 500 mg) 3 hours after the noon meal. All 3 of the subjects experienced severe flushing within 45 minutes to 1 hour after consuming the niacin tablet with 6 oz. water (placebo). After a 1 week washout, the same 3 subjects were given the same liquid xanthohumol formulation as in Example 1 above, in 6 oz. of water, but with 50 mg of xanthohumol added to water 60 minutes before consuming the same immediate release niacin as previously administered. Since immediate-release niacin is much faster absorbed than sustained-release niacin, all subjects without xanthohumol experienced severe flushing. There was a marked reduction in flushing when the subjects consumed 50 mg of xanthohumol 60 minutes prior to consuming the immediate release niacin, as indicated below in Tables 4 and 5. Flushing was assessed by both a subjective questionnaire and an observer questionnaire.

TABLE 4
Maximum Flushing Response, 500 mg, Immediate-release
Niacin, Incidence Post Initial Dosing - Subject

| | | | | | Very |
|---|---|---|---|---|---|
| | None | Mild | Moderate | Severe | Severe |
| Xanthohumol + | 2 | 1 | 0 | 0 | 0 |
| IR Niacin | | | | | |
| Placebo + | 0 | 0 | 0 | 3 | 0 |
| IR Niacin | | | | | |

TABLE 5
Maximum Flushing Response, 500 mg, Immediate-release
Niacin, Incidence Post Initial Dosing - Observer

| | | | | | Very |
|---|---|---|---|---|---|
| | None | Mild | Moderate | Severe | Severe |
| Xanthohumol + | 3 | 0 | 0 | 0 | 0 |
| IR Niacin | | | | | |
| Placebo + IR | 0 | 0 | 0 | 3 | 0 |
| Niacin | | | | | |

### Example 3

### Flushing Response Study

Five subjects were given capsules containing 250 mg of xanthohumol dissolved in a phospholipid emulsion (Phosal 53 MCT, Lipoid LLC, Newark, N.J.) that contained lecithin in caprylic/capric triglycerides, alcohol, glyceryl stearate, oleic acid, and ascorbyl palmitate. The subjects took this formulation 1 hour after the noon meal. Immediately after dinner, the subjects took 2 tablets containing 1,000 mg of sustained-release niacin (Slo-Niacin, Upsher-Smith Laboratories, Minnesota, Minn.). The subjects were asked to rate any nocturnal flushing by noting the questionnaire that night or the following morning immediately upon awaking. After a 1 week washout with no niacin or xanthohumol, the same subjects were given 2 tablets of sustained-release niacin from the same source as above, according to the same schedule, but with no xanthohumol (placebo given instead). Results were as follows:

TABLE 6
Maximum Flushing Response Incidence Post Dosing - Subjects

| | | | | | Very |
|---|---|---|---|---|---|
| | None | Mild | Moderate | Severe | Severe |
| Xanthohumol + | 4 | 1 | 0 | 0 | 0 |
| SR Niacin | | | | | |
| Placebo + | 0 | 0 | 3 | 2 | 0 |
| SR Niacin | | | | | |

Three of the five subjects who took the sustained-release niacin after the evening meal without xanthohumol were awakened by nocturnal flushing that lasted for about 45 minutes to 1 hour. This occurred about 4-5 hours after consuming the sustained-release niacin. None of the subjects who took the xanthohumol were awakened by nocturnal flushing. It appears that moderate to severe flushing is, in most cases, enough of a subjective annoyance to disturb sleep.

### Example 4

### Flushing Study with Reduced Titration Period

On day one, three subjects were first given 20 mg of xanthohumol in the water soluble formulation described in Example 1, two hours before being given 500 mg of sustained-release niacin (same formulation as in Example 1) 2 hours after the noon meal. None of the subjects flushed. On day two, the same 3 subjects were given 1,000 mg of sustained-release niacin and 20 mg of the water soluble xanthohumol at the same time. None of the subjects flushed. On day three, the 3 subjects were given 1,500 mg of the same sustained-release niacin at the same time and schedule after the noon meal, and with the same amount of xanthohumol. Again, none of the subjects flushed. The subjects continued for the next two days at the 1,500 mg dose of niacin with no flushing. Conversely, another 3 subjects who had not taken any niacin or xanthohumol for at least 2 weeks or more were given a water drink placebo two hours after the noon meal, and then given the same amount of niacin according to the same schedule. Two of the three subjects experienced some degree of flushing as assessed by a subjective questionnaire at about 3-4 hours post dose of the 500 mg sustained-release niacin on day one, and all three experienced moderate to severe flushing on day 2 at the 1,000 mg dose. In some cases, flushing occurred within the first two hours post dose of niacin. On day three, all three subjects took 1,500 mg of sustained-release niacin according to the same schedule. All three experienced extreme flushing within 2 to 4 hours post dose. This example illustrates the benefit of faster titration of niacin therapy, which enhances patient compliance, and speeds up the titration period of niacin therapy by many weeks (4-6 weeks is usually recommended to slowly increase the dose of niacin, usually starting with a dose of immediate release niacin of only 50 mg and working up to 1-2 grams per day over an extended period of time, or if sustained-release niacin, starting at 250 mg and slowly working up to 2 grams per day over a period of 4-6 weeks).

What is claimed is:

1. A pharmaceutical composition with hypolipemic or hypotryglyceridemic amounts of niacin having reduced capacity to provoke a flushing reaction in a subject, comprising a hypolipemic or hypotryglyceridemic amount of niacin, and a prenylflavonoid.

2. The composition of claim 1, wherein the prenylflavonoid is a prenylflavonone.

3. The composition of claim 1, wherein the prenylflavonoid is a prenylchalcone.

4. The composition of claim 1, wherein the prenylflavonoid is selected from the group consisting of xanthohumol, xanthogalenol, desmethylxanthohumol (2′,4′,6′,4-tetrahydrooxy-3-C-prenylchalcone), 2′,4′,6′,4-tetrahydrooxy-3′-C-geranylchalcone, dehydrocycloxanthohumol, dehydrocycloxanthohumol hydrate, 5′-prenylxanthohumol, tetrahydroxanthohumol, 4′-O-5′-C-diprenylxanthohumol, chalconaringenin, isoxanthohumol, 6-prenylnaringenin, 8-prenylnaringenin, 6,8-diprenylnaringenin, 4′,6′-dimethoxy-2′,4-dihydroxychalcone, 4′-O-methylxanthohumol, 6-geranylnaringenin, 8-geranylnaringenin, metabolites thereof, derivatives thereof, and combinations thereof.

5. The composition of claim 1, wherein the prenylflavonoid is xanthohumol, a xanthohumol metabolite, or a xanthohumol derivative.

6. The composition of claim 5, wherein the composition includes from 0.5 mg to 1000 mg of xanthohumol.

7. The composition of claim 5, wherein the composition includes from 5 mg to 250 mg of xanthohumol.

8. The composition of claim 5, wherein the composition includes from 10 mg to 50 mg of xanthohumol.

9. The composition of claim 1, wherein the prenylflavonoid is present in an amount suitable to at least partially alleviate flushing induced by the niacin in a majority of human subjects.

10. The composition of claim 1, wherein the prenylflavonoid is present in an amount suitable to completely alleviate flushing induced by the niacin in a majority of human subjects.

11. The composition of claim 1, wherein the amount of niacin is 50 mg to 4,000 mg.

12. The composition of claim 1, wherein the amount of niacin is 500 mg to 2,000 mg.

13. The composition of claim 1, wherein the niacin is in a sustained-release dosage form.

14. The composition of claim 1, wherein the amount of niacin is both a hypolipemic and a hypotryglyceridemic amount of niacin.

15. A method of suppressing cutaneous erythema in a patient to whom niacin is administered, comprising administering to the patient an amount of xanthohumol sufficient to reduce flushing prior to or concurrently with the administration of niacin.

16. The method of claim 15, wherein the amount of xanthohumol is from 1 mg to 500 mg, and the amount of niacin is from 50 mg to 4,000 mg.

17. The method of claim 15, wherein the amount of xanthohumol is from 5 mg to 50 mg, and the amount of niacin is from 500 mg to 2,000 mg.

18. The method of claim 15, wherein the niacin is in sustained-release dosage form.

19. The method of claim 15, wherein the xanthohumol is administered prior to the niacin.

20. The method of claim 19, wherein the xanthohumol is administered at least 1 hour prior to the niacin being administered so as to be released in or be absorbed by the patient prior to initial administration of the niacin.

21. The method of claim 15, wherein the xanthohumol is administered concurrently with the niacin.

22. A composition for treating dyslipidemia and hypertryclyceridemia, comprising an effective amount of niacin to treat dyalipidemia or hypertryglyceridemia, and xanthohumol in an amount effective to reduce cutaneous erythma caused by the effective amount of niacin.

23. The composition of claim 22, wherein the xanthohumol is in immediate release form and the niacin is in sustained-release or delayed release dosage form.

24. A pharmaceutical composition for administration of HDL elevating or hypotriglyceridemic amounts of niacin having reduced capacity to provoke a flushing reaction in a subject, comprising water soluble xanthohumol and sustained-release niacin.

25. A method of reducing or shortening the early titration phase of niacin dosing, wherein a first dose can be accelerated to at least 500 mg of sustained-release or immediate release niacin on a first day, and a subsequent titrations can be advanced to 1,000 mg of niacin on a second day and 1,500 mg on a third day, wherein, on the first day, a pre-dose of xanthohumol is administered at least 30 minutes prior to said first dose of niacin, and thereafter prior to or concurrently with higher doses of niacin.
